(19) United States
(12) Patent Application Publication
(10) Pub. No.: US 2010/0291184 A1
(43) Pub. Date: Nov. 18, 2010

(54) WOUND DRESSING WITH APERTURED COVER SHEET

(75) Inventors: Rachael Louise Clark, Skipton (GB); Margaret Stedman, North Yorkshire (GB); Sally Stephens, Skipton (GB)

Correspondence Address: MORGAN, LEWIS & BOCKIUS LLP, 1701 MARKET STREET, PHILADELPHIA, PA, 19103-2921, US

(73) Assignee: Systagenix Wound Management

(21) Appl. No.: 12/677,236

(22) PCT Filed: Sep. 10, 2008
(86) PCT No.: PCT/GB08/03075

§ 371 Date: Aug. 4, 2010

(30) Foreign Application Priority Data
Sep. 11, 2007 (GB) 0717698.5

(51) Int. Cl. A61K 9/70; A61K 33/38; A61P 31/00; A61P 17/02

(52) U.S. Cl. 424/445; 424/618

(57) ABSTRACT

A wound dressing comprising an antimicrobial absorbent layer containing silver, and an apertured sheet covering the antimicrobial absorbent layer, wherein the apertured sheet is formed from a liquid-impermeable sheet material having an array of apertures therein, said apertures having a mean effective diameter of from about 0.5 mm to about 2 mm, and wherein the percentage open area of the apertured sheet is from about 7% to about 25%. The selection of apertures in this range provides enhanced antimicrobial efficacy.

The present invention relates to a layered wound dressing comprising an antimicrobial absorbent layer and an apertured sheet covering the antimicrobial absorbent layer.

It is known that the adherency of wound dressings based on nonwoven textile absorbent layers can be reduced by providing a suitable apertured top sheet (wound contacting sheet) over the textile layer in the wound dressing. The top sheet reduces the tendency of the fibrous absorbent layer to adhere to the surface of a wound in use.

For example, GB-A-2074029 describes wound dressings having an absorbent layer of fibrous material and a top sheet of perforated polytetrafluoroethylene (PTFE) film covering the absorbent layer. The PTFE film provides a non-adherent wound contacting layer for the dressing.

GB-A-2085305 describes wound dressings in which a fibrous absorbent layer is entirely enclosed by a cellular plastics film having perforations of diameter 0.2 mm to 0.8 mm which have been produced by passing electrical discharges therethrough. The small apertures are said to result in low adherency of the wound contacting surface.

GB-A-1526778 describes wound dressings comprising an absorbent layer and a top sheet over the absorbent layer, wherein the top sheet is a plastic film having perforations that are tapered in cross-section, whereby the perforations promote the passage of wound fluid into the absorbent layer, but resist the flow of liquid from the absorbent layer back to the wound contacting surface.

EP-A-0275353 describes absorbent wound dressings comprising a top sheet formed of an elastomeric, soft, non-absorbent polyurethane film less than 50 micrometers thick, thermally bonded to a surface of an absorbent textile wound dressing, each perforation in the film having open areas equal to a circle having a diameter of 0.25 to 5 mm, the perforations being present in sufficient number, and so distributed, as to provide an open area in the range from 5% to 25% of the total area of the film. The use of a soft, elastomeric film is said to reduce tissue damage by the film. The selection of aperture size and open area is said to provide the desired balance of absorbency, non-adherency and mechanical properties.

GB-A-2392838 describes wound dressings comprising an absorbent layer and a top sheet having apertures that are initially blocked by a material that can be broken down by certain enzymes in wound fluid. The presence of these enzymes in the wound selectively activates the release of active agents from the absorbent layer by breaking down the material in the apertures and thereby increasing the effective open area of the top sheet. The apertures make up from 0.1 to 50% of the area of the top sheet, and the mean area of each aperture is from about 0.01 to about 10 mm².

The above art relates to wound dressings for the absorption of wound fluid. However, the use of a perforated top sheet potentially reduces the therapeutic effectiveness of more advanced wound dressings, in which the absorbent layer further comprises a therapeutic agent for delivery to the wound surface.

U.S. Pat. No. 4,715,857 describes a wound dressing comprising a fibrous, silver-loaded charcoal cloth layer enclosed in an envelope of a perforated plastics film having an aperture size of 0.05 to 0.5 mm. In such dressings, the use of such small perforations in the top sheet would be expected to inhibit the delivery of the therapeutic agent.

WO03/053584 describes wound dressing materials in the foam of a nonwoven fabric made up of a mixture of hydrogel-forming absorbent fibers and non-absorbent textile fibers, in which some of the non-absorbent textile fibers are coated with metallic silver (Ag⁰) to give the dressings antimicrobial properties. Dressings of this type comprising calcium alginate, carboxymethylcellulose (CMC) hydrogel-forming fibers blended with silver-coated nylon fibers are commercially available under the Registered Trade Mark SILVERCEL. Adherency remains a problem with hydrogel-containing dressings of this type. Moreover, the silver-coated fibers have a dark color, and it is therefore desirable to avoid shedding of these fibers into the wound under treatment because of the undesirable appearance of the dark fibers in the wound.

It would therefore be desirable to provide wound dressings of this type, wherein the shedding of silver-coated fibers into the wound is prevented, but with minimum loss of antimicrobial effectiveness.

The present invention provides a wound dressing comprising an antimicrobial absorbent layer containing silver, and an apertured sheet covering the antimicrobial absorbent layer, wherein the apertured sheet is formed from a liquid-impermeable sheet material having an array of apertures therein, said apertures having a mean effective diameter of from about 0.5 mm to about 2 mm, and wherein the percentage open area of the apertured sheet is from about 7% to about 25%.

The absorbent layer may comprise any of the materials conventionally used for absorbing wound fluids, serum or blood in the wound healing art, including gauzes, nonwoven fabrics, superabsorbents, hydrogels and mixtures thereof. Suitably, the absorbent layer comprises or consists essentially of a nonwoven textile fabric, for example a carded web of staple fibers. Suitably, the fabric comprises at least about 10 wt. % of hydrogel-forming absorbent fibers based on the dry weight of the fabric, for example, the fabric comprises at least about 20 wt. % of the hydrogel-forming fibers, for example from about 30 wt. % to about 50 wt. % of such fibers.

The term “hydrogel-forming fibers” refers to fibers that can absorb at least about twice their own weight of water, suitably at least about four times their own weight of water, to form a hydrogel. The fibers are normally insoluble in water. Suitable materials for the hydrogel-forming fibers include alginates, carboxymethylcelluloses, hydroxyethylcelluloses, polyacrylates, and hyaluronates. Preferred materials are calcium alginate and sodium carboxymethylcellulose and mixtures thereof.

Suitably, the fabric comprises at least about 10 wt. % based on the dry weight of the fabric of substantially non-water-absorbent textile fibers, and suitably it comprises at least about 20 wt. % of such fibers, for example from about 30 wt. % to about 60 wt. % of such fibers. Suitable non-absorbent textile fibers include polyamide fibers such as nylon fibers, polyolefin fibers, and viscose fibers.

Suitably, the absorbent layer is similar to those described in WO03/053584. That is to say, the absorbent layer comprises or consists essentially of a nonwoven fabric made up of a mixture of from about 10 wt. % to about 90 wt. % of hydrogel-forming absorbent fibers and from about 90 wt. % to about 10 wt. % of non-absorbent textile fibers, in which at least some of the non-absorbent textile fibers are coated with metallic silver (Ag⁰).

The term “fibers” herein generally refers to staple fibers, but it may refer to longer textile fibers. It does not refer to pulp fibers. In any case, the median length of the fibers used to form the fabric is generally at least about 10 mm. Suitably, the amount of silver in the fabric is from about 0.1% to about 10 wt. %, based on the dry weight of the fabric.

The absorbent layer contains silver as an antimicrobial agent. The silver may be metallic (Ag⁰) or ionic (Ag⁺), or a mixture thereof.

Suitably, the silver is metallic silver, suitably either colloidal metallic silver or a silver coating on one or more components of the absorbent layer. Suitably, the absorbent layer is a nonwoven textile layer wherein at least a fraction of the fibers in the absorbent fabric are coated with the silver to provide antimicrobial activity. Suitably, the silver coating is metallic silver applied to non-absorbent textile fibers of the fabric. The total amount of silver in the absorbent layer is typically from about 0.5 wt. % to about 10 wt. %, for example from about 1 wt. % to about 5 wt. % for metallic silver. These percentages are based on the dry weight of the absorbent layer.

In other embodiments, the silver comprises or consists essentially of ionic silver. Suitable silver compounds include silver oxide, silver chromate, silver allantbinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, and mixtures thereof. Suitably, the silver comprises or consists essentially of silver sodium hydrogen zirconium phosphate. Suitably, the silver is not present as silver sulfadiazine. The total amount of ionic silver is suitably from about 0.1 wt. % to about 2 wt. %, suitably from about 0.5 wt. % to about 1.5 wt. %. These percentages are based on the dry weight of the absorbent layer. Lesser amounts of silver could give insufficient antimicrobial effect.

In certain embodiments, the silver may be complexed to one or more anionic polymeric materials making up the absorbent layer. The term “complex” refers to an intimate mixture at the molecular scale, preferably with ionic or covalent bonding between the silver and the polymer. The complex preferably comprises a salt formed between an anionic polymer and Ag⁺. Suitably, the anionic polymer is a polycarboxylate. Suitably, the anionic polymer comprises an anionic polysaccharide or a polyacrylate. Suitable anionic polysaccharides include alginates, hyaluronates, pectins, carrageenans, xanthan gums, sulfated polysaccharides such as dermatan sulfate or sulfated dextrans, and carboxylated cellulose derivatives such as carboxymethyl celluloses. Especially suitable are alginates and oxidized celluloses such as oxidized regenerated cellulose, for example complexes of silver are formed with oxidized regenerated cellulose-containing fabrics or sponges as described in WO-A-2004024197. The silver may be introduced for example by treating the polymeric substrate material with a silver salt or compound dissolved or dispersed in water or an organic solvent such as ethanol, for example as described in WO-A-0243743.

The area of the absorbent layer is typically in the range of from 1 cm² to 200 cm², more suitably from about 4 cm² to about 150 cm². The shape of the absorbent layer may for example be circular, elliptical, square, rectangular, or other polygonal. Suitably, the basis weight of the absorbent layer is in the range of 50-1000 g/m², more suitably 100-500 g/m². The uncompressed thickness of the absorbent layer is suitably in the range of from about 0.5 mm to about 10 mm, more suitably about 1.5 mm to about 5 mm. The free (uncompressed) liquid absorbency measured for physiological saline is suitably in the range of 5 to 100 g/g at 25° C.

The thermoplastic film of the apertured cover sheet may be formed from substantially any thermoplastic film-forming polymer. Suitably, the polymer is conformable but not elastomeric. Suitably, the polymer is hydrophilic. Suitable polymers include, but are not limited to, polyethylene, polypropylene, polyester, polyamides such as nylons, fluoropolymers such as polyvinylidene fluoride (PVDF) or polytetrafluoroethylene (PTFE), and mixtures thereof. The currently preferred film forming thermoplastic polymer is ethylene methyl acrylate (EMA).

The apertured cover sheet may be textured. The term “textured” indicates that the film is patterned in relief, for example, patterned with protruding ridges or nubbles, for example by embossing. The texturing renders the film less adherent to a wound bed. The ridges or nubbles may be rounded, and may project by 0.1 to 1.5 mm above the median plane of the film surface, for example by 0.2 to 1.0 mm above the median plane of the film.

In certain embodiments the cover sheet is both textured and perforated by means of mesh perforation. In this method, the film is supported on a reticulated mesh surface and heated to its softening temperature. Suction is then applied through the mesh, or air is blown onto the film above the mesh, which results in impression of the mesh into the film and the formation of perforations in the film at the interstices of the mesh. Mesh perforation techniques are described in more detail in U.S. Pat. No. 3,054,148, the entire content of which is incorporated herein by reference.

The cover sheet should be as thin as possible, consistent with the need for physical integrity during manufacture and use. Typically, the sheet has a basis weight of from 1 to 500 g/m², for example from 10 to 200 g/m². Suitably, the apertures are open both before and during use. That is to say, they are not obstructed by a second material as described for example in GB-A-2392836.

As already noted, the size and density of the apertures in the top sheet is an important feature of the present invention. The apertures have an effective diameter of from about 0.5 mm to about 2.5 mm, and the percentage open area of the apertured sheet is from about 7% to about 25%. The term “mean effective diameter” refers to the diameter of a circle having equal area to the mean area of the apertures in the top sheet. The apertures may have non-circular shapes, but suitably substantially all of the apertures are circular. The apertures may be of uniform size, or there may be apertures of more than one size in the apertured sheet; for example, there may be a pattern of differently sized apertures. Not all of the apertures in the top sheet must have effective diameters in the specified range, so long as the mean effective diameter is in the specified range. However, suitably, at least about 50% of the apertures have effective diameters in the specified range, more suitably at least about 80%, and still more suitably substantially 100%. The telin “percentage open area” refers to the percentage of the area of the cover sheet that is taken up by the open area of the apertures.

The mean effective diameter of the apertures is suitably from about 0.8 mm to about 2 mm, for example from about 1 mm to about 2 mm, suitably from about 1.2 mm to about 1.8 mm. The percentage open area of the apertured sheet is suitably from about 15% to about 25%, for example from about 18% to about 25%. Suitably, the mean center-to-center distance between adjacent apertures is from about 2 mm to about 5 mm, for example from about 3 mm to about 5 mm.

A suitable cover sheet has apertures with mean effective diameter from about 1 mm to about 2 mm and percentage open area from about 17% to about 25%. More suitably, the cover sheet has apertures with mean effective diameter from about 1.1 mm to about 1.6 mm and percentage open area from about 19% to about 25%. For example, the cover sheet may have apertures with mean effective diameter of about 1.3 mm and percentage open area of about 22%.

The apertures may be any shape, but suitably they are substantially circular. The apertures are suitably substantially uniformly distributed over the top sheet, for example the apertures are suitably arranged in a regular array or pattern. Suitably, the cover sheet has from about 5 to about 50 apertures/cm², more suitably from about 10 to about 30 apertures/cm².

The apertured sheet material is positioned so that, in use, it lies between the wound contacting surface of the wound dressing and the absorbent layer. Suitably, the apertured sheet is the top sheet of the dressing, that is to say the material forms the wound contacting surface of the dressing. Suitably, the apertured sheet material is bonded directly to a wound facing surface of the absorbent layer by the application of heat and pressure to melt-bond the thermoplastic apertured sheet onto the absorbent layer. Apertured sheets according to the invention may be bonded in this way to both major surfaces of the absorbent layer.

The apertured sheet material can be formed into an envelope for the absorbent layer. The term “envelope” signifies that the front and back faces of the absorbent layer are substantially completely covered (substantially completely enclosed) by the apertured sheet. For example, two sheets of the apertured sheet may be located above and below the absorbent layer substantially covering the top and bottom faces of the absorbent layer, the two sheets being bonded along two or more edges to form the envelope. In certain embodiments the envelope is formed from a single piece of the apertured sheet that has been folded around the absorbent layer so that opposed longitudinal edges of the sheet overlap, the overlapping edges being bonded together in the overlapping region, typically with hot melt adhesive or by heat bonding, to form the envelope. Such envelopes may be made by minor modification of conventional form-fill-seal equipment, as described further below.

The wound dressing may comprise a backing sheet extending over the absorbent layer opposite to the wound facing side of the absorbent layer. Suitably, the backing sheet is larger than the absorbent layer such that a marginal region of width 1 mm to 50 mm, suitably 5 mm to 20 mm extends around the active layer to form a so-called island dressing. In such cases, the backing sheet is suitably coated with a pressure sensitive medical grade adhesive in at least its marginal region.

Suitably, the backing sheet is substantially liquid-impermeable. The backing sheet is suitably semipermeable. That is to say, the backing sheet is suitably permeable to water vapour, but not permeable to liquid water or wound exudate. Suitably, the backing sheet is also microorganism-impermeable. Suitable polymers for forming the backing sheet are well known in the wound dressing art.

The dressing may comprise further layers. For example, these layers may comprise further absorbent layers.

The wound facing surface of the dressing may be protected by a removable cover sheet. The cover sheet is normally formed from flexible thermoplastic material. Suitable materials include polyesters and polyolefins. Suitably, the adhesive-facing surface of the cover sheet is a release surface. That is to say, a surface that is only weakly adherent to the active layer and the adhesive on the backing sheet to assist peeling of the adhesive layer from the cover sheet. For example, the cover sheet may be formed from a non-adherent plastic such as a fluoropolymer, or it may be provided with a release coating such as a silicone or fluoropolymer release coating.

Suitably, the wound dressing according to the present invention is sterile and packaged in a microorganism-impermeable container.

Specific embodiments of the present invention will now be described further, by way of example, with reference to the accompanying drawings, in which:

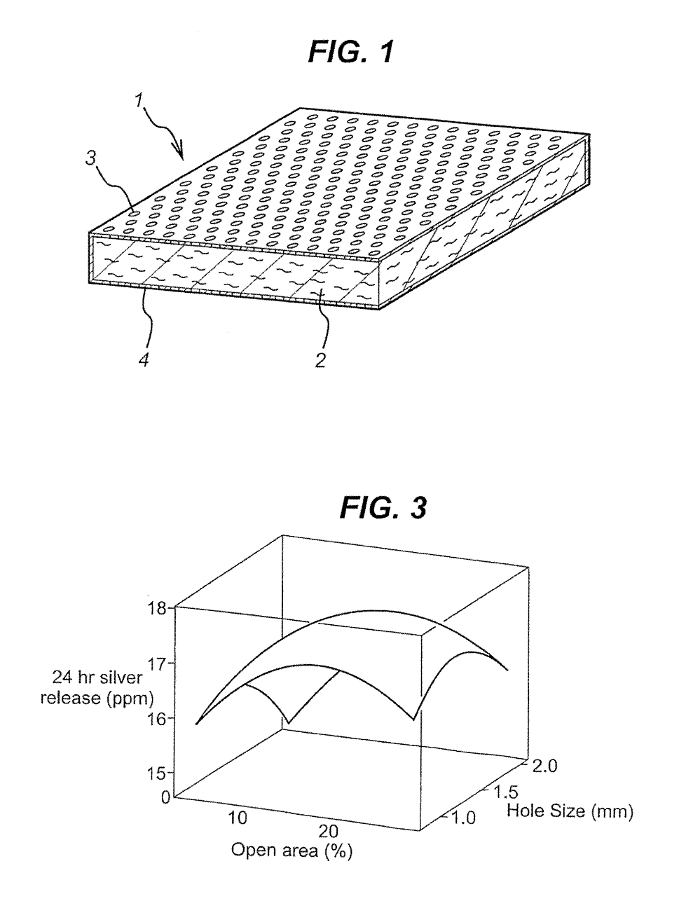

FIG. 1 shows a perspective view of a dressing according to the invention;

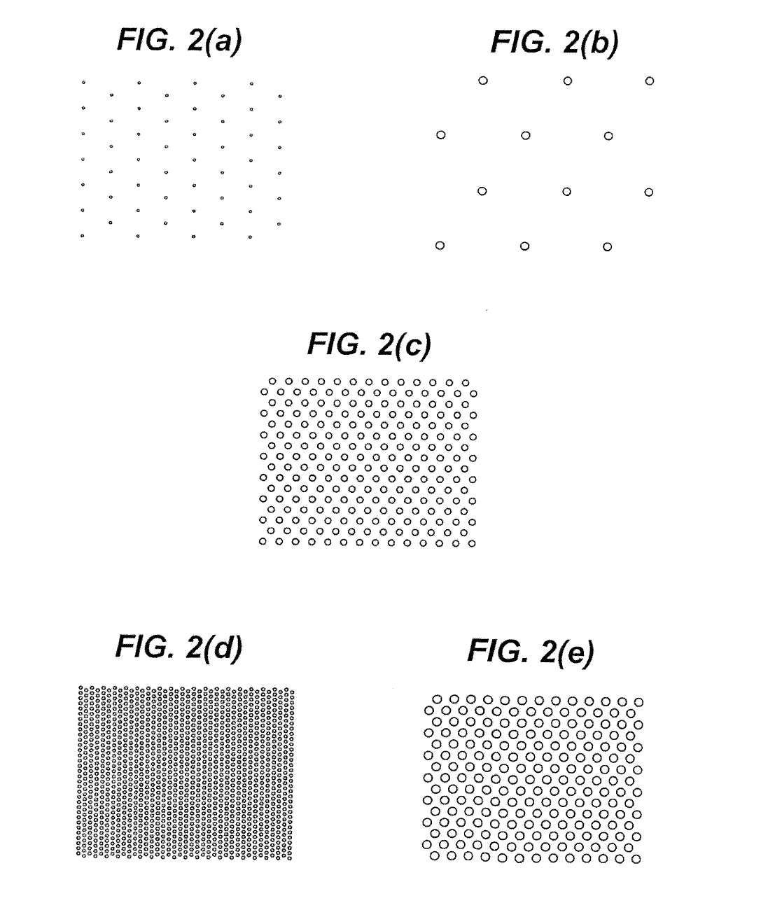

FIG. 2 shows schematic drawings of the aperture distribution in five different top sheets used for testing;

FIG. 3 shows a plot of measured silver release after 24 hours versus aperture size and open area of the top sheet for a dressing containing metallic silver;

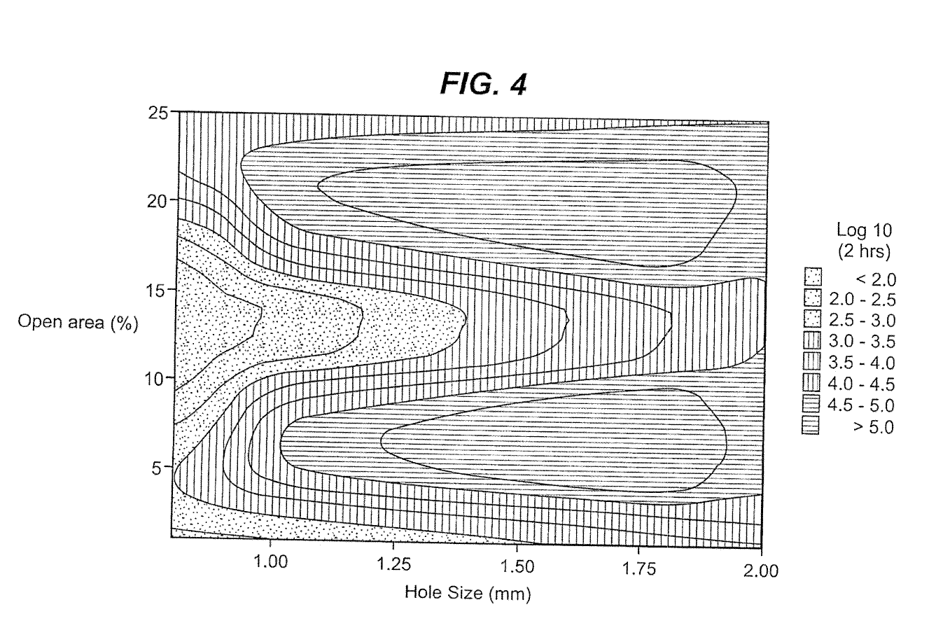

FIG. 4 shows a contour plot of measured log 10 bacterial reduction versus aperture size and open area of the top sheet for a dressing containing metallic silver

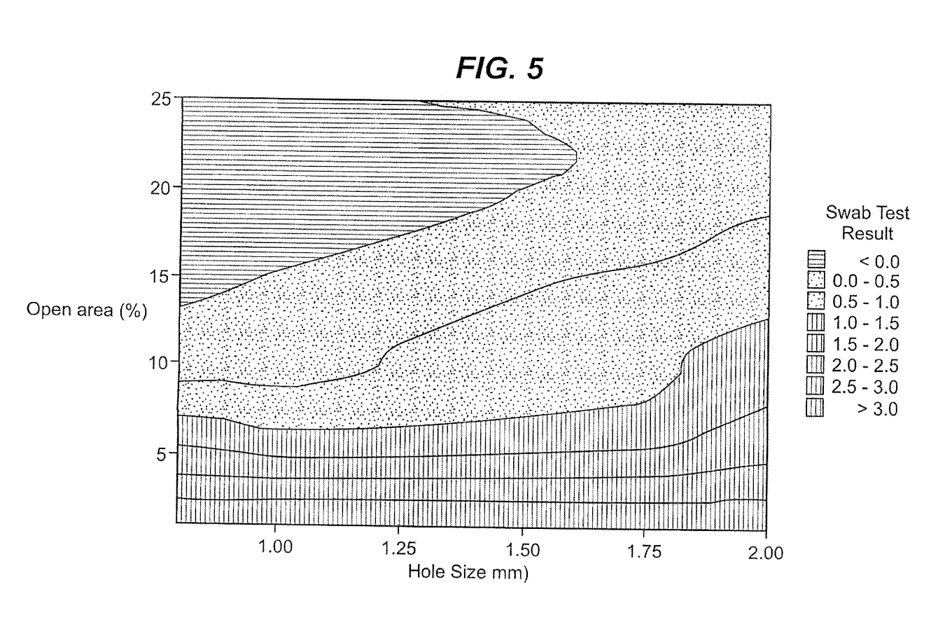

FIG. 5 shows a contour plot of swab test results in a three-day zone of inhibition test versus aperture size and open area of the top sheet for a dressing containing metallic silver

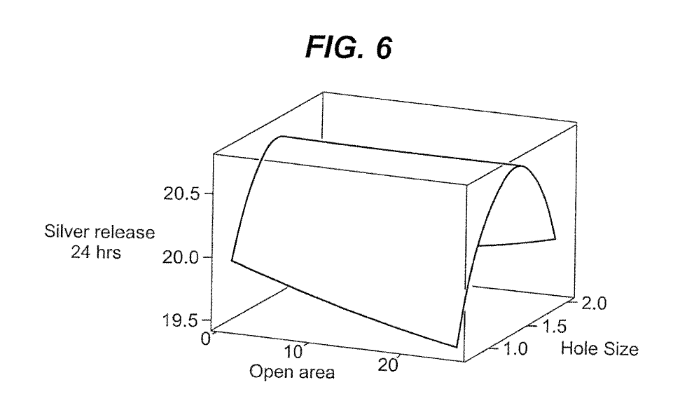

FIG. 6 shows a plot of measured silver release after 24 hours versus aperture size and open area of the top sheet for a dressing containing ionic silver; and

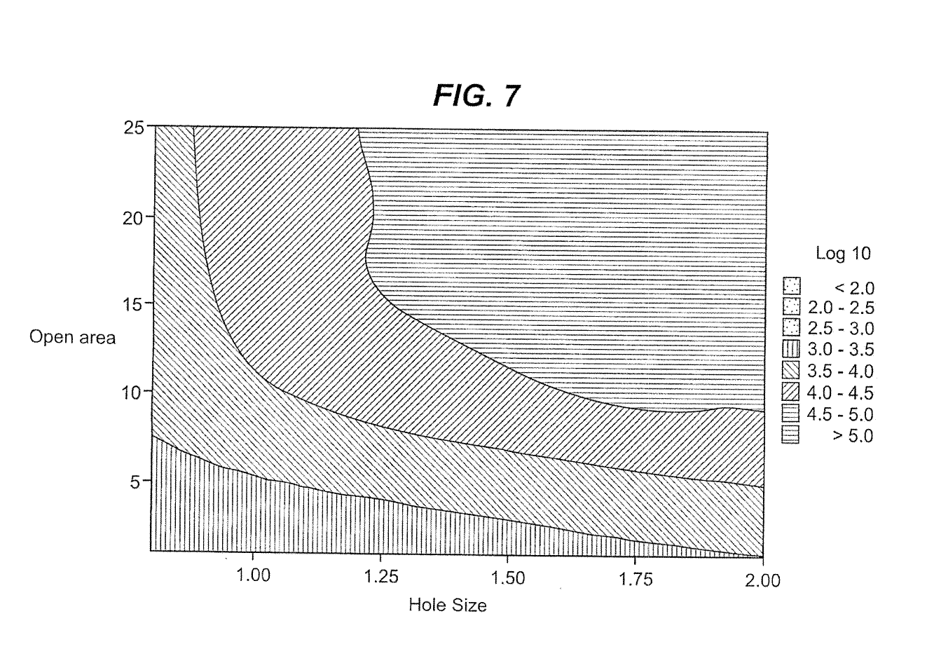

FIG. 7 shows a contour plot of measured log 10 bacterial reduction versus aperture size and open area of the top sheet for a dressing containing ionic silver

Referring to FIG. 1, the dressing 1 comprises an absorbent layer 2 containing metallic or ionic silver, having front and back sheets 3,4 of apertured EMA thermoplastic film bonded to the major surfaces thereof by application of heat and pressure.

### EXAMPLE 1

Wound dressings according to the invention containing metallic silver were made with the structure shown in FIG. 1. The absorbent layer is a calcium alginate needled felt dressing incorporating silver-coated nylon fibers. The composition is as follows, by weight: calcium alginate and carboxymethyl cellulose (CMC) fibers 60% and silver coated nylon 40%. The basis weight of the fabric layer is about 150 g/m², and the uncompressed thickness of the fabric layer is about 2 mm. The total silver content of the fabric is about 8 wt. %. The fabric layer is commercially available from Johnson & Johnson under the Registered Trade Mark SILVERCEL.

Dressings of this type were evaluated having top sheets with a range of hole sizes and percentage open areas, some of which are shown schematically in FIG. 2. Specifically, FIG. 2(a) represents 0.8 mm hole size, 1% open area; FIG. 2(b) represents 2 mm hole size, 1% open area; FIG. 2(c) represents 1.4 mm hole size, 13% open area; FIG. 2(d) represents 0.8 mm hole size, 25% open area; and FIG. 2(e) represents 2 mm hole size, 25% open area.

A total of 15 dressings of this type having differently perforated top sheets were tested. Hole sizes tested were, 0.8, 1.4 and 2.0 mm diameter, and percent open areas tested were 1%, 7%, 13%, 19% and 25%.

### EXAMPLE 2

Wound dressings according to the invention containing ionic silver were made with the structure shown in FIG. 1. The absorbent layer is a calcium alginate felt dressing incorporating an ionic silver complex. The composition is as follows, calcium alginate, carboxymethyl cellulose (CMC) fibers and silver sodium hydrogen zirconium phosphate. The total silver content of the fabric is about 0.5 wt. %. The fabric layer is commercially available from Laboratories Urgo, under the Registered Trade Mark Urgosorb Silver.

A total of 5 dressings of this type having differently perforated top sheets were tested. Hole sizes tested were, 0.8, 1.4 and 2.0 mm diameter, and percent open areas tested were 1%, 13%, and 25%.

### Procedure 1—Silver Release

Samples of the dressings of Examples 1 and 2 were immersed into a solution of simulated wound fluid (SWF), 0.013M Calcium Chloride, 0.2M Sodium Chloride and 0.04M Tris containing 2% Bovine Albumin at pH 7.5. Samples were gently agitated in an amount of SWF equivalent to 5 ml/2.5×2.5 cm². At a specific time point, for example 24 hours, a sample of fluid was removed and diluted in SWF and analysed against a silver standard Analysis was performed against a silver standard curve prepared in SWF using the Perkin Elmer Analyst 200 Atomic Absorption Spectrometer. Standards of known concentration were prepared in the same SWF as used for the samples. Specimen data for silver release in ppm are shown in Table 1 and are represented graphically in FIGS. 3 and 6.

TABLE 1
Silver release - Metallic Silver Dressings

| | | Percent open area | | | | |
|---|---|---|---|---|---|---|
| | | 1 | 7 | 13 | 19 | 25 |
| Hole size (mm) | 0.8 | 16.3 +/− 2.6 | 16.2 +/− 1.1 | 16.7 +/− 0.8 | 16.7 +/− 0.6 | 16.4 +/− 1.3 |
| | 1.4 | 16.4 +/− 1.3 | 18.0 +/− 1.0 | 17.7 +/− 1.4 | 17.4 +/− 1.4 | 17.4 +/− 1.1 |
| | 2 | 15.0 +/− 0.7 | 15.6 +/− 1.4 | 16.5 +/− 1.2 | 17.1 +/− 1.8 | 15.9 +/− 1.6 |

TABLE 2
Silver release - Ionic Silver Dressings

| | | Percent open area | | |
|---|---|---|---|---|
| | | 1 | 13 | 25 |
| Hole size (mm) | 0.8 | 19.6 +/− 0.37 | 20.41 +/− 0.88 | 19.21 +/− 0.53 |
| | 1.4 | 20.75 +/− 0.33 | 20.04 +/− 0.18 | 20.38 +/− 1.38 |
| | 2 | 20.00 +/− 0.83 | 18.73 +/− 0.61 | 20.5 +/− 0.95 |

It can be seen that there is a maximum in the silver release from the metallic silver dressings at a hole size of 1.4 mm and percentage open area 13%. At larger hole sizes and larger percentage open area the silver release is unexpectedly lower. The ionic silver dressings showed a maximum silver release at hole size of 1.4 mm, apparently independent of open area in the samples tested.

### Procedure 2—Inactivation of Bacteria in Suspension

Testing was performed using common wound pathogens, Staphylococcus aureus and Pseudomonas aeruginosa. The test dressings were immersed in a quantified bacterial suspension and then sampled to determine the numbers of challenge bacteria killed over a 120 minute test period. The log₁₀ bacterial inactivation was calculated.

The results are shown graphically in FIGS. 4 and 7 as contour plots. For the metallic silver dressings of FIG. 4, it can be seen that the log₁₀ bacterial inactivation increases sharply at hole sizes above about 0.8 mm, and shows two distinct maxima at percentage open areas about 7% and about 20%. For the ionic silver dressings of FIG. 7, it can be seen that the log₁₀ bacterial inactivation increases sharply at hole sizes above about 1.0 mm, and at open area above about 7%.

### Procedure 3—Three Day Zone of Inhibition Test and Swab Test

Testing was performed using agar plates inoculated with Staphylococcus aureus. Silver preferentially acts upon gram-negative bacteria (Pseudomonas aeruginosa) over gram-positive bacteria. (Staphylococcus aureus), and therefore Staphylococcus aureus was considered a more demanding test organism for the dressings of the invention. Dressings were transferred onto freshly inoculated agar plates on a daily basis for three days. The zone of inhibition was calculated by measuring the distance from the edge of the dressing to the edge of the clear zone surrounding the dressing. These measurements were made daily, prior to dressing transfer in order to determine the antimicrobial activity of the wound dressings, and whether this activity was sustained over three days. The mean±standard deviation was calculated for the zone of inhibition.

In addition, the ability of the dressing to prevent the growth of bacteria beneath the dressing was determined by taking a swab sample from the agar surface in direct contact with the dressing material and testing the swab for the presence of the bacteria.

The zone of inhibition (in mm) and swab results from dressings tested against Staphylococcus aureus are given in Tables 3 and 4. Positive results indicate growth of bacteria from swabs taken from the agar surface. One positive (1⁺) swab result is within the normal range of currently marketed anti-microbial dressings, therefore acceptable. Greater than one positive result upon swabbing is not acceptable for this test method. Ideally, no positive swab results (“Negative swab”) results should be obtained.

TABLE 3
Swab results - Metallic Silver

| | Hole Size (mm) | | |
|---|---|---|---|
| Open Area (%) | 0.8 | 1.4 | 2.0 |
| 1 | 3.13 ± 0.53 | — | 3.11 ± 0.44 |
| | 3 + swab | | 3 + swab |
| 7 | — | 3.21 ± 0.36 | — |
| | | 1 + swab | |
| 13 | 3.13 ± 0.23 | — | 3.61 ± 0.64 |
| | Negative swab | | 1 + swab |
| 19 | — | 3.58 ± 0.34 | — |
| | | Negative swab | |
| 25 | 2.82 ± 0.71 | — | 3.3 ± 0.40 |
| | Negative swab | | Negative swab |

TABLE 4
Swab results - Ionic Silver

| | Hole Size (mm) | | |
|---|---|---|---|
| Open Area (%) | 0.8 | 1.4 | 2.0 |
| 1 | 2.24 ± 0.71 | — | 2.83 ± 0.47 |
| | 2 + swab | | 3 + swab |
| 13 | — | 4.17 ± 0.45 | — |
| | | 2 + swab | |
| 25 | 4.89 ± 0.41 | — | 4.96 ± 0.56 |
| | Negative swab | | Negative swab |

It can be seen from the data that there is an optimal combination of aperture size and density for silver release and antimicrobial properties of the dressings.

The above embodiments have been described for the purpose of illustration only. Many other embodiments falling within the scope of the present invention will be apparent to the skilled reader.

1. A wound dressing comprising an antimicrobial absorbent layer containing silver, and an apertured sheet covering the antimicrobial absorbent layer, wherein the apertured sheet is formed from a liquid-impermeable sheet material having an array of apertures therein, said apertures having a mean effective diameter of from about 0.5 mm to about 2 mm, and wherein the percentage open area of the apertured sheet is from about 7% to about 25%.

2. A wound dressing according to claim 1, wherein the absorbent layer comprises or consists essentially of a nonwoven textile fabric comprising at least about 10 wt. % of hydrogel-forming absorbent fibers based on the dry weight of the fabric.

3. A wound dressing according to claim 1, wherein the silver comprises or consists essentially of metallic silver (Ag⁰)

4. A wound dressing according to claim 1, wherein the absorbent layer comprises or consists essentially of a nonwoven fabric made up of a mixture of from about 10 wt. % to about 90 wt. % of hydrogel-forming absorbent fibers and from about 90 wt. % to about 10 wt. % of non-absorbent textile fibers, in which at least some of the non-absorbent textile fibers are coated with metallic silver (Ag⁰).

5. A wound dressing according to claim 1, wherein the apertured sheet material is melt-bonded directly to a wound facing surface of the absorbent layer.

6. A wound dressing according to claim 1, wherein the silver comprises or consists essentially of ionic silver (Ag⁺).

7. A wound dressing according to claim 6, wherein the absorbent layer comprises an anionic polymer having said ionic silver complexed thereto.

8. A wound dressing according to claim 1, wherein the apertured sheet has apertures with mean effective diameter from about 1 mm to about 2 mm and percentage open area from about 17% to about 25%.

9. A wound dressing according to claim 1, wherein the apertured sheet has apertures with mean effective diameter from about 1.1 mm to about 1.6 mm and percentage open area from about 19% to about 25%.

10. A wound dressing according to claim 1, wherein the wound dressing is sterile.

11. A wound dressing according to claim 9, wherein the wound dressing is packaged in a microorganism impermeable container.
